(19) United States
(12) Patent Application Publication
(10) Pub. No.: US 2009/0239916 A1
(43) Pub. Date: Sep. 24, 2009

(54) NOVEL DRUG DISCOVERY TARGET AND MEDICINE ACTING ON THE SAME

(75) Inventors: Akito Tanaka, Ibaraki (JP); Akira Yamazaki, Osaka (JP); Akinobu Nakanishi, Tokyo (JP); Mikio Takeuchi, Ibaraki (JP); Masayuki Haramura, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Reverse Proteomics Research Institute Co., Ltd, Tokyo (JP)

(21) Appl. No.: 11/569,555

(22) PCT Filed: May 26, 2005
(86) PCT No.: PCT/JP2005/010105

§ 371 Date: Feb. 23, 2007

(30) Foreign Application Priority Data
May 26, 2004 (JP) 2004-156614

(51) Int. Cl. A61K 31/428; G01N 33/53; C12N 9/04; G01N 33/68; C07D 311/22; C07D 277/68; A61K 31/352; A61P 29/00

(52) U.S. Cl. 514/367; 435/7.8; 435/190; 436/86; 549/402; 548/165; 514/456

(57) ABSTRACT

The present invention provides a pharmaceutical composition comprising as an active ingredient a compound that specifically binds to MFP-2 or a functional fragment thereof and a screening method for the compound; the compound and a pharmaceutical composition comprising the same are highly useful as anti-inflammatory agents and anti-allergic agents.

### TECHNICAL FIELD

The present invention relates to a novel drug discovery target. More specifically, the present invention relates to a target molecule considered to be responsible for the efficacy of an anti-inflammatory agent or an anti-allergic agent, such as tiaramide, Intal and derivatives thereof. The present invention also relates to a compound that specifically binds to the target molecule.

### BACKGROUND ART

With the decoding of the base sequences of the human genomes, subjects of research have been shifting to genomic drug discovery and search and identification of drug discovery targets. As such, subjects include the identification of targets considered to be responsible for the efficacy of tiaramide and derivatives thereof, or Intal and derivatives thereof, which have been commonly used as anti-inflammatory and anti-allergic agents.

To date, as a mechanism for tiaramide, which is classified as a non-acidic (basic) NSAIDs (non-steroidal anti-inflammatory drugs), an action to suppress the function of the prophlogistic factor histamine or serotonin at inflammatory sites and hence to suppress airway constriction has mainly been proposed (Chiryoyaku Manual 2004, p76; Arzneimittelforschung, 1972, Apr. 22 (4), pp. 724-732), and the suppression of TXA2 production has been shown to be a mechanism for the airway constriction suppression (see, for example, Folco G C et al., Arzneimittelforschung, Germany, 1982, 32(9), pp. 1092-1095).

Also, Intal, known as an anti-allergic drug, is known to have suppressant action of the release of chemical mediators such as histamine and SRS-A from mast cells that occurs in relation to antigen-antibody reactions (Chiryoyaku Manual 2004, p301), and the like; to date, regarding the action mechanisms thereof, available reports include the inhibition of the secretion of histamine and the like with phospholipase A stimulation (see, for example, Orr T S. and Cox J S, Nature, U K, 1969, 223 (202), pp. 197-198), as well as the involvement of a protein involved in the calcium channel (see, for example, Mazurek N. et al., Proceedings of the National Academy of Sciences USA., USA, 1984, 81 (21), pp. 6841-6845) and the involvement of intracellular protein kinase (see, for example, Theoharides T C. et al., Science, USA, 1980, 207 (4426), pp. 80-82) and the like.

However, despite enormous efforts, identification of targets with which these drugs interact directly, and whose pharmacological activity be explained, has been an unresolved problem. For this reason, and also because of difficulty in developing an efficient method of screening for compounds surpassing these compounds in terms of efficacy, it has been extremely difficult to create a drug surpassing these compounds.

Whereas ordinary NSAIDs exhibit cyclooxygenase inhibition as their essential anti-inflammatory action, elucidation of this different type of effective anti-inflammatory mechanism is expected to lead to the discovery of a drug having high therapeutic effect that supplements the therapeutic effects of existing drugs such as cyclooxygenase inhibitors, which produce severe adverse reactions such as gastrointestinal disorder, or that replace them; therefore, elucidation of the true mechanism has been awaited. For anti-allergic agents as well, the same elucidation has been awaited.

It is an object of the present invention to identify a target considered to be responsible for the efficacy of tiaramide and derivatives thereof, and Intal and derivatives thereof, as anti-inflammatory and anti-allergic agents, and to provide a screening method for a compound useful in the treatment of a disease such as an inflammatory disease or an allergic disease using the target, and a compound obtained by the screening.

### DISCLOSURE OF THE INVENTION

The present inventors diligently investigated to solve the above-described problems in search of a protein to which Intal and tiaramide bind specifically in common. As a result, the present inventors found that a protein called MFP-2 (multifunctional protein 2; Accession No. P70523) specifically binds to Intal and tiaramide, confirmed that the protein is a sufficient drug discovery target to explain the anti-inflammatory and anti-allergic actions of these derivatives, developed a screening method for a compound useful in the treatment of an inflammatory disease or an allergic disease using such a target or cells expressing the target, and thus developed the present invention.

Accordingly, the present invention relates to the following:

[1] A pharmaceutical composition comprising as an active ingredient a compound that specifically binds to a protein having the amino acid sequence of SEQ ID NO:2.
[2] A pharmaceutical composition comprising, as an active ingredient, a compound that specifically binds to a protein, which is characterized by having an amino acid sequence resulting from the deletion, substitution or addition of one or more amino acids in the amino acid sequence of SEQ ID NO:2 and binding to a compound of the formula (1) and/or a compound of the formula (2).

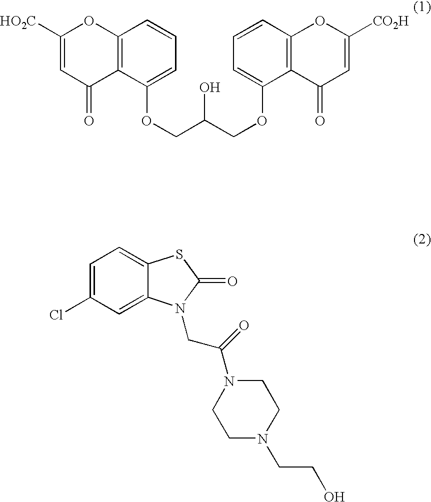

[3] The pharmaceutical composition described in [1] or [2] above, which is for the treatment of a disease selected from the group consisting of an inflammatory disease and an allergic disease.
[4] A pharmaceutical composition comprising as an active ingredient a compound that specifically binds to MFP-2.
[5] A pharmaceutical composition comprising as an active ingredient a compound that regulates the expression of MFP-2.
[6] A pharmaceutical composition comprising as an active ingredient a compound that regulates the activity of MFP-2.
[7] The pharmaceutical composition described in any of [4] to
[6] above, which is for the treatment of a disease selected from the group consisting of an inflammatory disease and an allergic disease.
[8] A method for screening for a compound useful for the treatment of a disease selected from the group consisting of an inflammatory disease and an allergic disease, which comprises the following steps of
(1) bringing MFP-2 or a functional fragment thereof into contact with a test compound,
(2) determining whether or not the test compound specifically binds to MFP-2 or a functional fragment thereof, and
(3) selecting a test compound that specifically binds to MFP-2 or a functional fragment thereof in the step (2) above.
[9] A method for screening for a compound useful for the treatment of a disease selected from the group consisting of an inflammatory disease and an allergic disease, which comprises the following steps of
(1) bringing a protein having the amino acid sequence of SEQ ID NO:2 or a functional fragment thereof into contact with a test compound,
(2) determining whether or not the test compound specifically binds to the protein or a functional fragment thereof, and
(3) selecting a test compound that specifically binds to the protein or a functional fragment thereof in the step (2) above.
[10] A method for screening for a compound useful for the treatment of a disease selected from the group consisting of an inflammatory disease and an allergic disease, which comprises the following steps of
(1) bringing a protein, which is characterized by having an amino acid sequence resulting from the deletion, substitution or addition of one or more amino acids in the amino acid sequence of SEQ ID NO:2 and binding to a compound of the formula (1) and/or a compound of the formula (2), or a functional fragment thereof, into contact with a test compound,

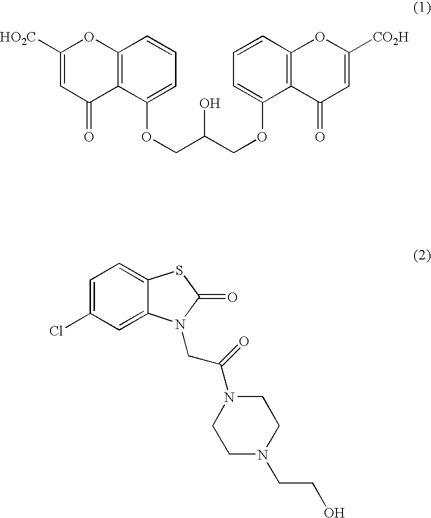

(2) determining whether or not the test compound specifically binds to the protein or a functional fragment thereof, and
(3) selecting a test compound that specifically binds to the protein or a functional fragment thereof in the step (2) above.
[11] A compound useful for the treatment of a disease selected from the group consisting of an inflammatory disease and an allergic disease, obtained by the screening method described in any of [8] to [10] above.
[12] A compound represented by the general formula (1) or the general formula (II) or a pharmaceutically acceptable salt thereof:

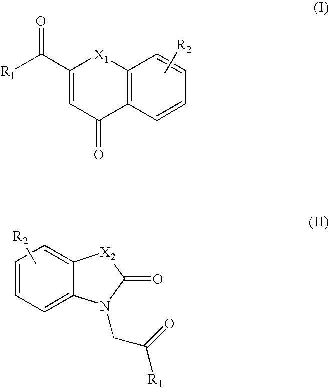

(wherein X₁ is O, NH, NR′″, S, SO or SO₂;

### X₂ is O, NH, NR′″, S, —CH₂CH₂— or —CH═CH—;

### R₁ is H, OH, OR′, NH₂, NHR′ or NR′R″;

R₂ is an optionally substituted saturated or unsaturated chain-structured hydrocarbon group, an optionally substituted alkoxy group, an optionally substituted amino group, an optionally substituted carboxyl group, an optionally substituted amide group or a halogen atom;
R′, R″ and R′″ are the same or different and each is an optionally substituted saturated or unsaturated chain-structured hydrocarbon group;
provided that R₁ is NR′R″, R′ and R″ may bind together to form a ring in cooperation with the adjoining nitrogen atom), but the compounds represented by the formula (1) and the formula (2) are excluded.

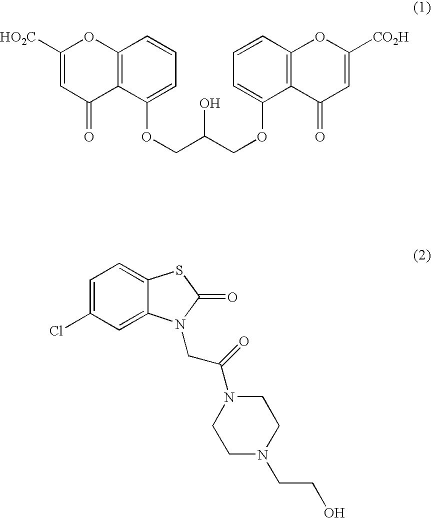

[13] A pharmaceutical composition comprising as an active ingredient the compound described in [12] above or a pharmaceutically acceptable salt thereof.
[14] The pharmaceutical composition described in [13] above, which is for the treatment of a disease selected from the group consisting of an inflammatory disease and an allergic disease.

### BRIEF DESCRIPTION OF THE DRAWINGS

FIG. 1-1 is a drawing showing the degrees of homology of MFP-2 among rats, mice, and humans.

FIG. 1-2 is a drawing showing the degrees of homology of MFP-2 among rats, mice, and humans (continued from FIG. 1-1).

FIG. 2 is a drawing showing the results of an examination of the specificity of the MFP-2 binding of each compound. Onto a solid phase carrier with a compound that specifically binds to the MFP-2 immobilized thereon, MFP-2 binds quickly with a first immobilizing resin treatment.

### DETAILED DESCRIPTION OF THE INVENTION

MFP-2 (multifunctional protein 2) has also been reported as rat-derived proteins such as multifunctional beta-oxidant protein 2, peroxisomal (Accession No. P70523), peroxisomal multifunctional enzyme type 2 (MFE-2), D-bifunctional protein (DBP), and 17-beta-hydroxysteroid dehydrogenase 4 (17-beta-HSD4) (all Accession No. P97852). In mice, the same protein has been reported as hydroxysteroid 17-beta dehydrogenase (Accession No. Q9 DBM3), MFE-2, DBP, 17-beta-HSD4 (all Accession No. P51660) and the like; as human-derived proteins, the same protein has been reported as MFE-2, DBP, 17-beta-HSD4 (all Accession No. P51659) and the like. MFP-2 is a protein having a number of unlimited various synonyms. Structurally, MFP-2 is speculated to have many physiological actions because it has a short-chain dehydrogenase/reductase domain in the vicinity of the N-terminus thereof and a MaoC-like dehydratase domain and a sterol-binding domain-like domain on the C-terminus side. For example, MFP-2 is known to exhibit 2-enoyl-CoA hydratase and D-3-hydroxyacyl-CoA dehydrogenase activities, and to be involved in the beta oxidation of fatty acids.

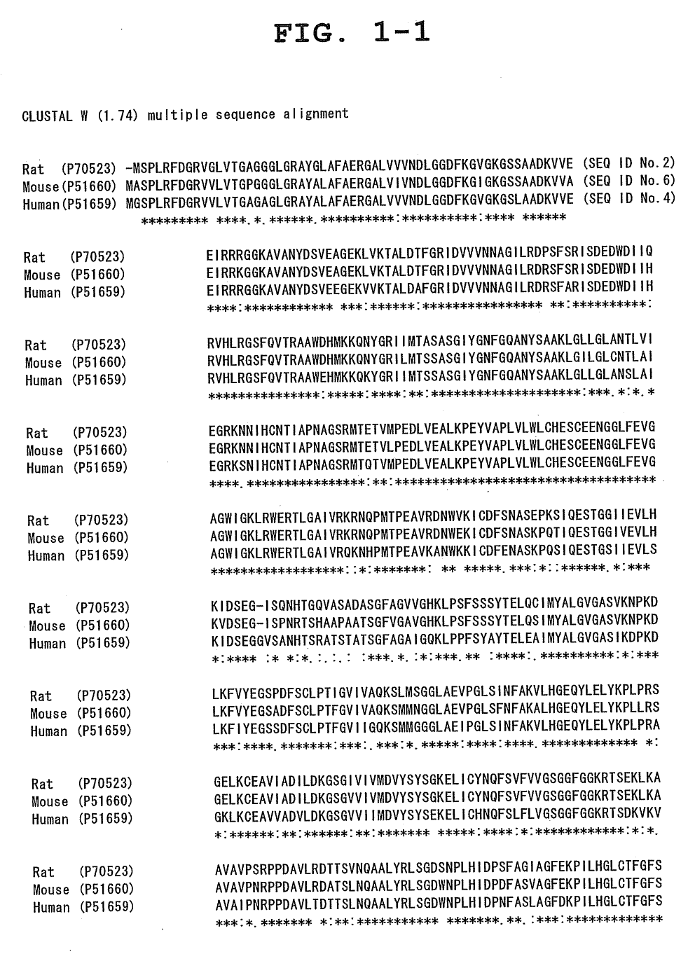

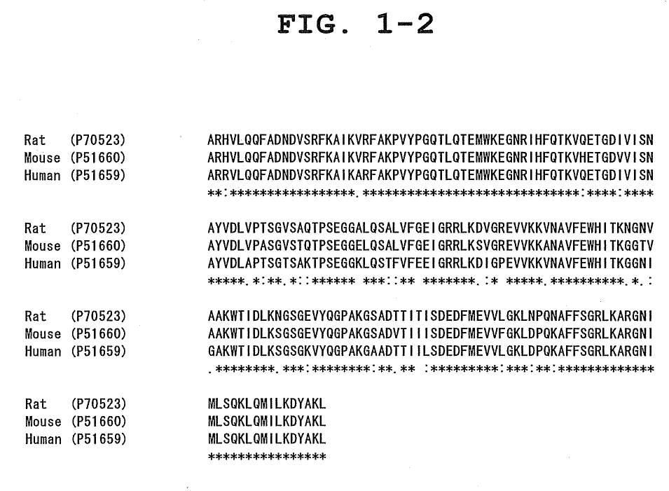

More specifically, a protein comprising 734 amino acids, shown by the amino acid sequence of SEQ ID NO:2 (Accession No. P70523), can be mentioned as an example; as long as it binds specifically to Intal, tiaramide, derivatives thereof, or to a compound exhibiting anti-inflammatory action or anti-allergic action by a mechanism similar to that for these compounds, that is, acts as a target molecule, MFP-2 may be a protein having an amino acid sequence resulting from the deletion, substitution or addition of one or more amino acids in the amino acid sequence of SEQ ID NO:2. Therefore, although MFP-2 exhibits somewhat different amino acid sequences among species (see FIG. 1), the MFP-2 of any species is an MFP-2 that can be used in the present invention. For example, the amino acid sequence of SEQ ID NO:2 is derived from the rat, but human MFP-2, which is shown by the amino acid sequence of SEQ ID NO:4, is also encompassed in the MFP-2 of the invention of this application.

More specifically, the MFP-2 that can be used in the present invention does not always need to be shown only by the amino acid sequence of SEQ ID NO:2, as long as it is a protein, which is characterized by binding to a compound of the formula (1) and/or a compound of the formula (2),

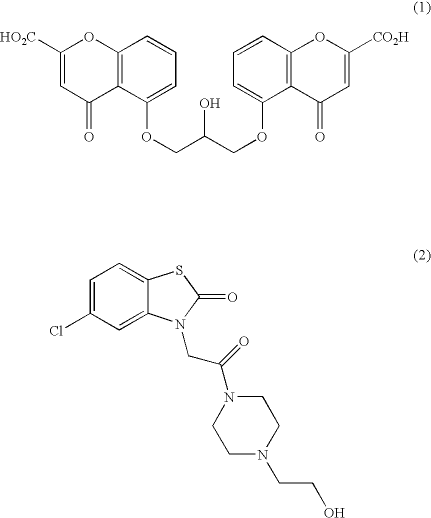

and it may be a protein having the amino acid sequence of SEQ ID NO:2, or a protein having an amino acid sequence resulting from the deletion, substitution or addition of one or more amino acids in the amino acid sequence of SEQ ID NO:2. More specifically, the MFP-2 is a protein shown by an amino acid sequence having a homology of 60% or more, 70% or more, 80% or more, preferably 90% or more, and more preferably 95% or more, to the amino acid sequence of SEQ ID NO:2, and is further preferably a protein (polypeptide) comprising 40 continuous amino acids or more, preferably 70 continuous amino acids or more, and particularly preferably 100 continuous amino acids or more, in the amino acid sequence of SEQ ID NO:2.

As used herein, “homology” means the degree of sequence correlation between two polypeptide sequences. Homology can easily be calculated. A large number of methods of measuring the homology between two polypeptide sequences are known, and the term “homology” (also called “identity”) is obvious to those skilled in the art. Ordinary methods used to measure the homology of two sequences include, but are not limited to, those disclosed in Martin, J. Bishop (Ed.), Guide to Huge Computers, Academic Press, San Diego (1994); Carillo, H. & Lipman, D., SIAM J. Applied Math., 48:1073 (1988) and the like. As a preferable method for measuring the homology, one designed to obtain the largest matching portion between the two sequences tested can be mentioned. As such a method, one assembled in a computer program can be mentioned. Preferable computer programming methods for measuring the homology between two sequences include, but are not limited to, the GCG program package (Devereux, J. et al., Nucleic Acids Research, 12(1):387 (1984)), BLASTP, FASTA and the like; methods known in the art can be used.

Furthermore, in the present invention, the MFP-2 may be a fragment of MFP-2, as long as it is characterized by binding to a compound of the formula (1) and/or a compound of the formula (2), provided that it can serve in common as a target for a series of anti-inflammatory agents and anti-allergic agents such as Intal and tiaramide; such a fragment is hereinafter also referred to as a functional fragment of MFP-2. To increase the accuracy, it is desirable that the MFP-2 should bind to both a compound of the formula (1) and a compound of the formula (2).

All these embodiments are included in the MFP-2 in the present invention unless otherwise specified.

To determine whether or not the MFP-2 or a functional fragment thereof can be used in the present invention, a compound of the formula (1) and/or a compound of the formula (2), preferably both a compound of the formula (1) and a compound of the formula (2), are used; these compounds are already known compounds, and are commercially available or can be produced according to a known technology.

“specifically bind” is exemplified by the relation of a specific receptor to an agonist or an antagonist, the relation of an enzyme to a substrate, and the relation of, for example, an FK506-binding protein (target molecule) to FK506 (ligand), a steroid hormone receptor to a steroid hormone (e.g., dexamethason and glucocorticoid receptor), HDAC to the anticancer agent trapoxin, and the like, and can be confirmed as numerical values of Kd, Ka and the like by competitive experiments and the like. As described in Examples below, this can also be confirmed by a visual means such as electrophoresis, in addition to representation as specific numerical values.

The present invention provides a pharmaceutical composition comprising, as an active ingredient, a compound that specifically binds to MFP-2 (a protein having the amino acid sequence of SEQ ID NO:2, a protein, which is characterized by having the amino acid sequence of SEQ ID NO:2 and binding to a compound of the formula (1) and/or a compound of the formula (2), and the like) and a functional fragment thereof. Such a compound exhibits anti-inflammatory action and/or anti-allergic action by binding to MFP-2, which is a novel target for an anti-inflammatory agent or an anti-allergic agent, to regulate the expression of MFP-2 and/or regulate the activity thereof. An “inflammatory disease” is an exogenous or endogenous acute or chronic disease; in the case of acute disease, the five major signs of fever, reddening, swelling, pain and functional impairment are evident. For example, pain and inflammation following surgery in various departments and those following trauma, or pain and inflammation in arthritis, lumbago, cervico-omo-brachial syndrome, intrapelvic inflammation, damage of the soft birth canal, breast engorgement, herpes zoster, erythema exsudativum multiforme, cystitis, epididymitis, anterior ocular inflammation, pericoronitis of wisdom tooth, or after tooth extraction and the like, or diseases such as acute upper airway inflammation can be mentioned. As examples of the “allergic disease”, diseases such as bronchial asthma, allergic rhinitis, and atopic dermatitis can be mentioned.

Furthermore, according to the finding obtained in the present invention that MFP-2 can serve as a novel drug discovery target for anti-inflammatory agents, anti-allergic agents and the like, a compound that does not bind directly to MFP-2 but does act directly or indirectly thereon to regulate the expression or activity of MFP-2 can also be said to be useful for the treatment of inflammatory disease and allergic disease.

As examples of the compound (substance) that regulates the expression or activity of MFP-2, a DNA that encodes MFP-2, a vector incorporating a DNA that encodes MFP-2, MFP-2 protein and the like can be mentioned.

A compound capable of binding to MFP-2 or a functional fragment thereof, like Intal or tiaramide, exhibits excellent anti-inflammatory action and/or anti-allergic action on various mammals, and is therefore useful as a therapeutic agent for inflammatory disease such as an anti-inflammatory agent, and as a therapeutic agent for allergic disease such as an anti-allergic agent (subject diseases are the same as described above).

As examples of the compound contained in the present invention as an active ingredient, specifically, a compound represented by the general formula (1) or the general formula (II) or a pharmaceutically acceptable salt thereof can be mentioned.

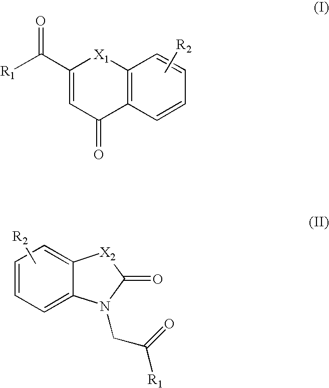

(wherein X₁ is O, NH, NR′″, S, SO or SO₂;

### X₂ is O, NH, NR′″, S, —CH₂CH₂— or —CH═CH—;

### R₁ is H, OH, OR′, NH₂, NHR′ or NR′R″;

R₂ is an optionally substituted saturated or unsaturated chain-structured hydrocarbon group, an optionally substituted alkoxy group, an optionally substituted amino group, an optionally substituted carboxyl group, an optionally substituted amide group or a halogen atom;
R′, R″ and R′″ are the same or different and each is an optionally substituted saturated or unsaturated chain-structured hydrocarbon group;
provided that R₁ is NR′R″, R′ and R″ may bind together to form a ring in cooperation with the adjoining nitrogen atom), but the compounds represented by the formula (1) and the formula (2) are excluded.

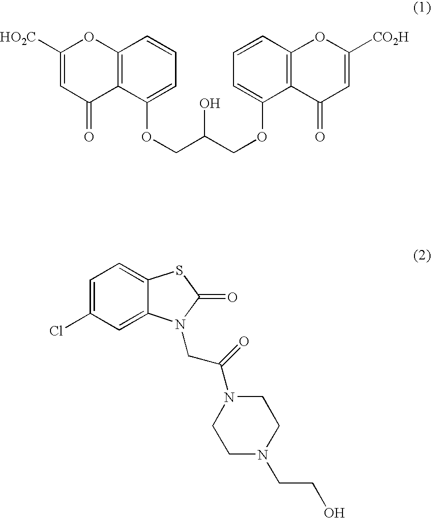

“A saturated or unsaturated chain-structured hydrocarbon group” denotes, for example, a linear or branched chain-structured hydrocarbon group having 1 to 10 carbon atoms, and the like; specifically, for example, an alkyl group, an alkenyl group, an alkynyl group and the like can be mentioned. Of these groups, an alkyl group is particularly preferable. As examples of the “alkyl group”, an alkyl group having 1 to 10 carbon atoms, such as methyl, ethyl, n-propyl, isopropyl, butyl, isobutyl, sec-butyl, tert-butyl, n-pentyl, isopentyl, neopentyl, n-hexyl, and isohexyl, and the like can be mentioned. As examples of the “alkenyl group”, an alkenyl group having 2 to 10 carbon atoms, such as vinyl, 1-propenyl, allyl, isopropenyl, 1-butenyl, 2-butenyl, 3-butenyl, isobutenyl, and sec-butenyl, and the like can be mentioned. As examples of the “alkynyl group”, an alkynyl group having 2 to 10 carbon atoms, such as ethynyl, 1-propynyl, and propargyl, and the like can be mentioned.

The substituent for “an optionally substituted saturated or unsaturated chain-structured hydrocarbon group” is not subject to limitation; for example, a saturated or unsaturated cyclic hydrocarbon group (described below), a saturated or unsaturated heterocyclic group (described below), a halogen atom (described below), a cyano group, a nitro group, a hydroxyl group, an optionally substituted carboxyl group (described below), a substituted amide group (described below), an optionally substituted lower alkyl group (described below), an optionally substituted aroxy group, an optionally substituted amino group (described below), an optionally substituted alkoxy group and the like can be mentioned. These substituents substitute on the chain-structured hydrocarbon group, as long as the substitution is chemically acceptable. However, provided that the number of substituents is two or more, they may be the same or different.

“An alkoxy group” means a linear or branched alkoxy group having 1 to 6 carbon atoms; specifically, methoxy group, ethoxy group, n-propoxy group, isopropoxy group, n-butoxy group, isobutoxy group, sec-butoxy group, tert-butoxy group, n-pentyloxy group, isopentyloxy group, tert-pentyloxy group, neopentyloxy group, 2-pentyloxy group, 3-pentyloxy group, n-hexyloxy group, 2-hexyloxy group and the like can be mentioned.

The substituent for “an optionally substituted alkoxy group” is not subject to limitation; for example, a saturated or unsaturated cyclic hydrocarbon group (described below), a saturated or unsaturated heterocyclic group (described below), a halogen atom (described below), a cyano group, a nitro group, a hydroxyl group, an optionally substituted carboxyl group (described below), a substituted amide group (described below), an optionally substituted lower alkyl group (described below), an optionally substituted aroxy group, an optionally substituted amino group (described below), an optionally substituted alkoxy group and the like can be mentioned. These substituents substitute on the alkoxy group, as long as the substitution is chemically acceptable. However, provided that the number of substituents is two or more, they may be the same or different.

As “an optionally substituted amino group”, an amino group optionally substituted by a lower alkyl group (described below), a lower alkanoyl group (for example, an alkanoyl group having 1 to 6 carbon atoms, such as formyl, acetyl, and propionyl) and the like, and the like can be mentioned.

As “an optionally substituted carboxyl group”, a carboxyl group optionally substituted by a lower alkyl group (described below), a lower alkanoyl group (for example, an alkanoyl group having 1 to 6 carbon atoms, such as formyl, acetyl, and propionyl) and the like, and the like can be mentioned.

As “an optionally substituted amide group”, an unsubstituted amide group, a substituted amide [N-substituted amide group or N,N′-di-substituted amide group; specifically, an amide group substituted by a lower alkyl group (described below), and the like] can be mentioned.

As “a halogen atom”, fluorine, chlorine, bromine, and iodine can be mentioned.

As the ring that R′ and R″ may bind together to form in cooperation with the adjoining nitrogen atom, a saturated or unsaturated heterocyclic group (described below) comprising a nitrogen atom can be mentioned, and the ring is optionally substituted by a halogen atom (described above), a carboxyl group, a substituted amide group (described above), an optionally substituted lower alkyl group (described below) and the like.

As examples of “a saturated or unsaturated heterocyclic group”, a 5- to 6-membered monocyclic group comprising one to two nitrogen atoms, a 5- to 6-membered monocyclic group comprising one to two nitrogen atoms and one oxygen atom or one sulfur atom, a 5-membered monocyclic group comprising one oxygen atom or one sulfur atom, a bicyclic group comprising one to four nitrogen atoms and resulting from the condensation of a 6-membered ring and a 5- or 6-membered ring, and the like can be mentioned; specifically, for example, pyridyl, thienyl, oxaziazolyl, imidazolyl, thiazolyl, isothiazolyl, oxazolyl, isoxazolyl, furyl, pyrrolyl, quinolyl, quinazolinyl, purinyl, pyrazolyl, thiophenyl and the like can be mentioned. The heterocyclic group is optionally substituted by a substituent such as a saturated or unsaturated cyclic hydrocarbon group (described below), a saturated or unsaturated heterocyclic group (described above), a halogen atom (described above), a cyano group, a nitro group, an oxo group, an optionally substituted carboxyl group (described above), a substituted amide group (described above), an optionally substituted lower alkyl group (described below), an optionally substituted amino group (described above), or an optionally substituted alkoxy group (described above); these substituents substitute on the heterocyclic group, as long as the substitution is chemically acceptable. Provided that the number of substituents is two or more, they may be the same or different.

“A lower alkyl group” denotes, for example, a linear, branched or cyclic alkyl group having 1 to 6 carbon atoms; specifically, methyl, ethyl, n-propyl, isopropyl, butyl, isobutyl, sec-butyl, tert-butyl, pentyl, hexyl, cyclopropyl, cyclobutyl and the like can be mentioned. As the “substituent” in “an optionally substituted lower alkyl group”, a carboxyl group, a substituted amide group (having the same definition as described above), a cyano group, a hydroxyl group, a halogen atom (described above) and the like can be mentioned.

As examples of “an aroxy group”, a 6-membered monocyclic group (for example, phenyl group) across an oxygen atom can be mentioned; specifically, for example, phenoxy and the like can be mentioned. The monocyclic group is optionally substituted by a substituent such as a saturated or unsaturated cyclic hydrocarbon group (described below), a saturated or unsaturated heterocyclic group (described above), a halogen atom (described above), a cyano group, a nitro group, an optionally substituted carboxyl group (described above), a substituted amide group (described above), an optionally substituted lower alkyl group (described below), an optionally substituted amino group (described above), or an optionally substituted alkoxy group (described above); these substituents substitute on the cyclic group, as long as the substitution is chemically acceptable. Provided that the number of substituents is two or more, they may be the same or different. Furthermore, the monocyclic group may have condensed with a saturated or unsaturated cyclic hydrocarbon group (described below) or with a saturated or unsaturated heterocyclic group (described above), to form a condensed ring. As the condensed ring, indene, naphthalene, fluorene, phenanthrene, anthracene, indole, isoindole, benzofuran, benzothiophene, indolizine, chromene, quinoline, isoquinoline, indazole, quinazoline, cinnoline, quinoxaline, phthalazine and the like can be mentioned.

“A saturated or unsaturated cyclic hydrocarbon group” denotes a saturated or unsaturated cyclic hydrocarbon group having 3 to 18 carbon atoms; specifically, for example, an alicyclic hydrocarbon group, an aromatic hydrocarbon group and the like can be mentioned.

As examples of the “alicyclic hydrocarbon group”, a monocyclic or condensed polycyclic group comprising 3 to 10 carbon atoms, specifically, a cycloalkyl group, a cycloalkenyl group and a bicyclic or tricyclic condensed ring thereof with an aryl group having 6 to 14 carbon atoms (for example, benzene and the like) or the like, and the like can be mentioned. As examples of the “cycloalkyl group”, a cycloalkyl group having 3 to 6 carbon atoms, such as cyclopropyl, cyclobutyl, cyclopentyl, and cyclohexyl can be mentioned; as examples of the “cycloalkenyl group”, a cycloalkenyl group having 3 to 6 carbon atoms, such as cyclopropenyl, cyclobutenyl, cyclopentenyl, and cyclohexenyl, and the like can be mentioned.

As examples of the “aromatic hydrocarbon group”, a monocyclic aromatic hydrocarbon group comprising 6 to 18 carbon atoms, a condensed polycyclic aromatic hydrocarbon group and the like can be mentioned; specifically, an aryl group having 6 to 14 carbon atoms, such as phenyl, 1-naphthyl, 2-naphthyl, 2-indenyl, and 2-anthryl, can be mentioned.

The cyclic hydrocarbon group is optionally substituted by a substituent such as a saturated or unsaturated cyclic hydrocarbon group (described above), a saturated or unsaturated heterocyclic group (described above), a halogen atom (described above), a cyano group, a nitro group, an oxo group, an optionally substituted carboxyl group (described above), a substituted amide group (described above), an optionally substituted lower alkyl group (described above), an optionally substituted amino group (described above), and an optionally substituted alkoxy group (described above); these substituents substitute on the cyclic hydrocarbon group, as long as the substitution is chemically acceptable. However, provided that the number of substituents is two or more, they may be the same or different.

The compound of the present invention, represented by the general formula (I) or the general formula (II), can be produced by applying various commonly known synthetic methods by means of the characteristics based on the basic skeleton thereof or the kind of substituent. For example, alkylation, acylation, amination, imination, halogenization, reduction, oxidation, condensation and the like can be mentioned, and reactions or methods in common use in the art can be utilized.

A compound capable of binding to MFP-2 or a functional fragment thereof, such as the compound of the present invention, represented by the general formula (I) or the general formula (II), exhibits excellent anti-inflammatory action and anti-allergic action in mammals such as monkeys, horses, cattle, sheep, dogs, cats, rabbits, mice, rats, and guinea pigs, including humans, and is therefore useful for the treatment of a disease selected from the group consisting of an inflammatory disease and an allergic disease.

The compound used in the present invention, which binds specifically to MFP-2 and exhibits anti-inflammatory action and/or anti-allergic action (hereinafter also referred to as the compound of the present invention), may have formed a pharmaceutically acceptable salt; as the salt, acid addition salts, for example, inorganic acid salts (for example, hydrochlorides, sulfates, hydrobromates, phosphates and the like), organic acid salts (for example, acetates, trifluoroacetates, succinates, malates, fumarates, propionates, citrates, tartrates, lactates, oxalates, methanesulfonates, p-toluenesulfonates and the like) and the like can be mentioned.

Note that the compound of the present invention or a salt thereof may be a solvate such as a hydrate.

When the compound of the present invention is used as a therapeutic drug for a disease selected from the group consisting of an inflammatory disease and an allergic disease, it is prepared as an ordinary pharmaceutical preparation and administered orally or parenterally.

For oral administration, the compound of the present invention can be administered in a dosage form in common use in the art. For parenteral administration, the compound of the present invention can be administered in a dosage form such as a topical preparation (transdermal preparation and the like), a rectal preparation, an injection, or a nasal preparation.

As examples of the oral preparation or rectal preparation, capsules, tablets, pills, powders, drops, cachets, suppositories, liquids and the like can be mentioned. As examples of the injection, a sterile solution or suspension and the like can be mentioned. As examples of the topical preparation, creams, ointments, lotions, transdermal preparations (ordinary patches, matrices) and the like can be mentioned.

The above-described dosage forms can be formulated along with a pharmaceutically acceptable excipient and additive by a technique commonly performed in the art. As the pharmaceutically acceptable excipient and additive, carriers, binders, flavoring agents, buffering agents, thickeners, colorants, stabilizers, emulsifiers, dispersing agents, suspending agents, antiseptics and the like can be mentioned.

As examples of pharmaceutically acceptable carriers, magnesium carbonate, magnesium stearate, talc, sugar, lactose, pectin, dextrin, starch, gelatin, gum tragacanth, methylcellulose, sodium carboxymethylcellulose, low-melting-point waxes, cacao butter and the like can be mentioned.

Furthermore, the tablets can be prepared as tablets with ordinary coatings, for example, sugar-coated tablets, enteric coated tablets, film-coated tablets, and double-layered tablets or multilayered tablets if necessary. The powders are formulated into preparations along with a pharmaceutically acceptable base for powders. As the base, talc, lactose, starch and the like can be mentioned. The drops can be formulated into preparations along with an aqueous or non-aqueous base and one kind or more of pharmaceutically acceptable diffusing agents, suspending agents, solubilizers and the like. The capsules can be produced by filling therein an active ingredient compound, along with a pharmaceutically acceptable carrier. The compound can be mixed with a pharmaceutically acceptable excipient and filled in the capsules, or filled without an excipient. The caches can also be produced in the same manner. When the present invention is prepared as a suppository, it is formulated into preparations by a commonly used technique along with a base such as a vegetable oil (castor oil, olive oil, peanut oil and the like), a mineral oil (petrolatum, white petrolatum and the like), a wax, or a partially synthesized or totally synthesized glycerine fatty acid ester.

As the liquid for injection, solutions, suspensions, emulsions and the like can be mentioned. For example, aqueous solutions, water-propylene glycol solutions and the like can be mentioned. The liquid can also be produced in the form of a solution of polyethylene glycol and/or propylene glycol that may contain water.

A liquid suitable for oral administration can be produced by adding an active ingredient compound to water and, if required, adding a colorant, flavoring agent, stabilizer, sweetener, solubilizer, thickener and the like. A liquid suitable for oral administration can also be produced by adding the compound, along with a dispersing agent, to water to increase the viscosity. As examples of the thickener, pharmaceutically acceptable natural or synthetic rubbers, resins, methylcellulose, sodium carboxymethylcellulose, known suspending agents and the like can be mentioned.

As the topical preparation, the above-described liquids, as well as creams, aerosols, sprays, dusting powders, lotions, ointments and the like can be mentioned. The above-described topical preparation can be produced by mixing an active ingredient compound and pharmaceutically acceptable diluent and pharmaceutically acceptable carrier. Ointments and creams are prepared by, for example, adding a thickener and/or a gelling agent to an aqueous or oily base. As examples of the base, water, liquid paraffin, vegetable oils and the like can be mentioned. As examples of the thickener, soft paraffin, aluminum stearate, cetostearyl alcohol, propylene glycol, polyethylene glycol, lanolin, hydrogenated lanolin, beeswax and the like can be mentioned. To the topical preparation, an antiseptic such as methyl hydroxybenzoate, propyl hydroxybenzoate, chlorocresol, or benzalkonium chloride, and a bacterial growth inhibitor can be added. A lotion can be prepared by adding one or more kinds of pharmaceutically acceptable stabilizers, suspending agents, emulsifiers, diffusing agents, thickeners, colorants, flavoring agents and the like to an aqueous or oily base.

Dosage and frequency of administration vary depending on the kind of compound used, symptoms, age and body weight of patients, dosage form and the like, and are set as appropriate according to them.

The present invention also enables screening for a compound useful for the treatment of a disease such as an inflammatory disease or an allergic disease, with specific bindability to MFP-2 or a functional fragment thereof (the definitions for the individual terms are as described above) as an index. Here, MFP-2 or a functional fragment thereof can be used as a purified or unpurified protein (polypeptide) or a (functional) fragment thereof, and can be used in the state of being expressed in cells. MFP-2 or a (functional) fragment thereof can be acquired by using as appropriate a known technique such as (1) a method comprising isolation and purification from a cell culture or tissue, as a starting material, producing MFP-2 or a (functional) fragment thereof, (2) a method comprising chemical synthesis, or (3) a method comprising purification from cells manipulated by gene recombination technology and the like to express MFP-2 or a (functional) fragment thereof.

Isolation and purification of the MFP-2 or a (functional) fragment thereof of the present invention can, for example, be performed as described below. That is, the MFP-2 or a (functional) fragment thereof is extracted and purified by a known method from a tissue expressing MFP-2 or a (functional) fragment thereof, or a culture obtained by culturing cells expressing MFP-2 or a (functional) fragment thereof in an appropriate liquid medium. For the extraction and purification, known methods are used as appropriate depending on the fraction wherein the desired product is present.

Specifically, the extraction and purification are performed as described below. First, a tissue or culture is directly subjected to a conventional method such as filtration or centrifugation, and the tissue or cells or the supernatant is recovered. If the desired protein has been accumulated in the cells, the recovered cells are suspended in an appropriate buffer solution, and a surfactant is added at an appropriate concentration to solubilize the membrane. As the surfactant, sodium dodecyl sulfate (SDS), cetyltrimethylammonium bromide (CTAB) and the like can be mentioned. Since these exhibit potent protein denaturing action, it is preferable to use a gently acting nonionic surfactant, for example, Triton X-100 and the like, to ensure that the protein is folded to show biological activity. Next, the crude extract obtained is treated in the presence of a surfactant if required, using commonly used methods in combination as appropriate, to isolate and purify the protein or a functional fragment thereof. As such methods, methods based on differences in solubility, such as salting-out and solvent precipitation; methods based on differences in molecular weight, such as dialysis, ultrafiltration, gel filtration, and SDS-PAGE; methods based on electric charge, such as ion exchange chromatography; methods based on specific affinity, such as affinity chromatography; methods based on differences in hydrophobicity, such as reverse phase high performance liquid chromatography; methods based on differences in isoelectric point, such as isoelectric focusing; and the like can be mentioned. More specifically, the protein or a functional fragment thereof can be separated and purified by commonly used methods, for example, concentration under reduced pressure, lyophilization, extraction with conventionally used solvents, pH adjustment, treatment with conventionally used adsorbents such as anion exchange resin or cation exchange resin, and nonionic adsorption resin, crystallization, recrystallization and the like.

Production of the MFP-2 or a (functional) fragment thereof of the present invention by chemical synthesis can be performed by, for example, synthesis or semi-synthesis based on the amino acid sequence information shown by SEQ ID NO:2 using a peptide synthesizer.

Also, when the MFP-2 or a (functional) fragment thereof is acquired from cells manipulated to express the same by gene recombination technology and the like, the specific procedures are performed as described below.

First, an expression vector that functionally carries the gene encoding MFP-2 or a functional fragment thereof is prepared.

The gene that encodes MFP-2 or a functional fragment thereof may be obtained by any method. For example, a complementary DNA (cDNA) prepared from an mRNA, a genomic DNA prepared from a genomic library, a chemically synthesized DNA, a DNA obtained by amplification by the PCR method with an RNA or DNA as a template, and a DNA constructed by appropriately combining these methods, and the like are included. For example, a DNA comprising all or a portion of a DNA substantially comprising the base sequence shown by SEQ ID NO:1 (Accession No. X94978), particularly the base sequence shown by the base numbers 28 to 2232, a DNA comprising all or a portion of a DNA substantially comprising the base sequence shown by SEQ ID NO:3 (Accession No. X87176), particularly the base sequence shown by the base numbers 49 to 2259, a DNA comprising all or a portion of a DNA substantially comprising the base sequence shown by SEQ ID NO:5 (Accession No. X89998), particularly the base sequence shown by the base numbers 13 to 2220, and the like can be mentioned. Also, a technology for substituting or deleting an optionally chosen base in the above-described base sequence (for example, in vitro mutagenesis, site-directed mutagenesis and the like) can also be utilized.

As used herein, “a DNA substantially comprising” means, in addition to the above-described DNAs comprising a particular base sequence, a DNA comprising a base sequence capable of hybridizing to the above-described DNAs comprising a particular base sequence under stringent conditions (in the present invention, these conditions refer to conditions under which a DNA having a homology of about 60% or more, preferably about 80% or more, and more preferably about 90% or more, in terms of base sequence can hybridize; stringency can be controlled by changing the temperature, salt concentration and the like as appropriate during the hybridization reaction and washing). Stringent conditions can be calculated on the basis of the desired homology, the length of oligonucleotide and the like by applying them to appropriate calculation formulas utilized in the art. For example, hybridization at 42° C. and washing treatment at 42° C. with a buffer solution containing 1×SSC and 0.1% SDS, hybridization at 65° C. and washing treatment at 65° C. with a buffer solution containing 0.1×SSC and 0.1% SDS, and the like can be mentioned.

An expression vector that functionally comprises a gene encoding MFP-2 or a functional fragment thereof can be obtained by inserting the DNA obtained into a plasmid vector, phage vector or the like capable of retaining replication or autonomous replication in various hosts of prokaryotic cells and/or eukaryotic cells by means of an appropriate restriction endonuclease site.

As used herein, “functionally” means that the gene (DNA) is arranged to allow transcription in a host cell matching with the vector, and to allow the production of the protein encoded thereby. Preferably, the expression vector is a vector having an expression cassette wherein a promoter region, an initiation codon, a gene encoding MFP-2 or a functional fragment thereof, a stop codon and a terminator region are continuously arranged. For transformant selection, it is preferable that a selection marker gene be further contained.

For example, when a mammalian cell is transformed, a plasmid comprising a promoter and a polyadenylation signal both of an animal virus, for example, SV40, RSV, MMLV and the like, joined to each other via a restriction endonuclease site, preferably a multicloning site, wherein a selection marker gene derived from a plasmid such as pSV2-neo or pSV2-dhfr (neomycin resistance gene, dihydrofolate reductase and the like) has been inserted, can be used.

The host cell is not subject to limitation, as long as it matches with the expression vector used, and is transformable; various cells in common use in the technical field of the present invention, such as natural cells or an artificially established line of recombinant cells and the like, can be utilized. Specifically, bacteria such as Escherichia coli and Bacillus subtilis, fungi such as yeast, animal cells or insect cells and the like can be mentioned as examples. Preferably, mammalian cells, particularly rat-derived cells, hamster-derived cells (CHO, BHK and the like), mouse-derived cells (COP, L, C127, Sp2/0, NS-1, NIH T3 and the like), monkey-derived cells (COS1, COS3, COS7, CV1, Velo and the like) and human-derived cells (Hela, diploid fibroblast-derived cells, myeloma cells, Namalwa, Jurkat cells and the like) can be mentioned.

Introduction of an expression vector to a host cell can be performed using a conventionally known method. For example, when the expression vector is introduced to a mammalian cell, the calcium phosphate co-precipitation method, the protoplast fusion method, the microinjection method, the electroporation method, the lysosome method and the like can be mentioned.

MFP-2 or a functional fragment thereof can also be produced by culturing a transformant comprising an expression vector prepared as described above. The medium preferably contains a carbon source and inorganic or organic nitrogen source required for the growth of the host cell (transformant). As examples of the carbon source, glucose, dextrin, soluble starch, sucrose and the like can be mentioned; as examples of the nitrogen source, ammonium salts, nitrates, amino acids, corn steep liquor, peptone, casein, meat extract, soybean cake, potato extract and the like can be mentioned. If desired, other nutrients [for example, inorganic salts (calcium chloride, sodium dihydrogen phosphate, magnesium chloride and the like), vitamins, antibiotics (tetracycline, neomycin, kanamycin, ampicillin and the like)] may be contained.

The cultivation is performed by a method known in the art. The cultivation conditions are conditions enabling the expression of the protein; for example, temperature, medium pH and cultivation time are chosen as appropriate so that the protein is produced in a large amount.

For example, when the host is an animal cell, as examples of the medium, a minimum essential medium (MEM) containing about 5 to 20% fetal calf serum (FCS), Dulbecco's modified Eagle medium (DMEM), RPMI-1640 medium, 199 medium and the like can be used. The pH of the medium is preferably about 6 to 8, the cultivation is normally performed at 30 to 40° C. for about 15 to 72 hours, and the culture may be aerated or agitated as necessary.

The MFP-2 or a functional fragment thereof of the present invention can be collected from the culture obtained from the above-described cultivation in the same manner as the aforementioned extraction, isolation, and purification from cells or tissue expressing MFP-2 or a functional fragment thereof.

Contact treatment of the MFP-2 or functional fragment thereof thus obtained and a test compound can be performed in accordance with a binding experiment commonly performed in the art. Specifically, in cases where MFP-2 or a functional fragment thereof or a test compound is immobilized to a solid phase carrier, a solution comprising the test compound is brought into contact with the solid phase carrier when the MFP-2 or a functional fragment thereof is immobilized, and a solution comprising the MFP-2 or a functional fragment thereof (a purified protein solution or a crudely purified protein solution such as cell extract or tissue extract) is brought into contact with the solid phase carrier when the test compound is immobilized to the solid phase carrier. The column method, the batch method and the like can be utilized.

The step for determining whether or not the test compound binds specifically to MFP-2 or a functional fragment thereof can be changed as appropriate depending on how the step for bringing the test compound into contact with MFP-2 or a functional fragment thereof has been performed; for example, when using a column packed with a solid phase carrier (for example, bead resin) immobilized with the test compound, MFP-2 molecules bind onto the solid phase carrier with the subsequent addition of a solution (sample) comprising MFP-2 or a functional fragment thereof, provided that there is specific affinity between the two (do not bind in the absence of specific affinity). It is also possible to dissociate the bound MFP-2 or a functional fragment thereof from the solid phase by a treatment such as altering the polarity of the buffer solution or further adding the test compound in excess, and then identify, or to extract with a surfactant and the like while remaining in a state bound to the test compound on the solid phase, and then identify. As the method of identification, specifically, known techniques such as electrophoresis, immunoblotting and immunoprecipitation, which employ immunological reactions, chromatography, mass spectrometry, amino acid sequencing, and NMR, or combinations of these methods can be used. By determining whether or not MFP-2 or a functional fragment thereof is captured onto the solid phase or contained in the column through fraction, or the extent thereof and the like, a judgment is made as to whether or not the test compound is capable of binding specifically to MFP-2, and a binding compound is selected.

Also, this step may be automated. For example, it is also possible to directly read data on various molecules obtained by two-dimensional electrophoresis, and identify the molecules on the basis of existing databases.

Furthermore, when MFP-2 or a functional fragment thereof is used in the state of being expressed in cells, it is also possible to determine the presence or absence of binding of MFP-2 or a functional fragment thereof and the test compound, and the degree of binding, by making use of various labeling techniques such as RI labeling and fluorescence labeling. “Contact of MFP-2 or a functional fragment thereof and a test compound” in the screening method of the present invention also includes this mode. The contact conditions of cells and a test compound are set as appropriate depending on factors such as the cells used and the expression status of MFP-2 or a functional fragment thereof in the cells. Also, whether or not MFP-2 or a functional fragment thereof is expressed in the cells is preferably confirmed in advance using an antibody and the like.

### EXAMPLES

The present invention is hereinafter described in more detail by means of the following Examples, which, however, are not to be construed as limiting the scope of the invention. Also, the compounds, reagents and the like used are commercially available or can be prepared on the basis of known reports and the like, unless otherwise specified.

### Example 1

### Synthesis of Intal-Immobilized Resin

### (1) Synthesis of 1,3-bis(2-carboxychromon-5-yloxy)-2-hydroxypropane

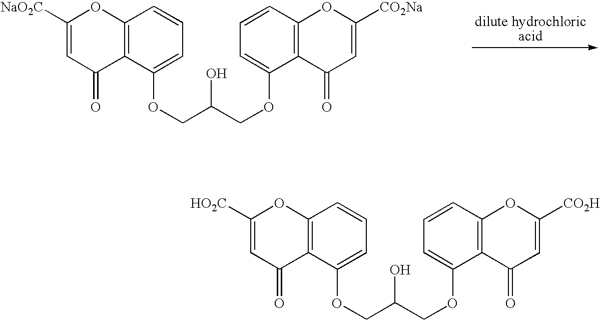

The disodium salt of 1,3-bis(2-carboxychromon-5-yloxy)-2-hydroxypropane (Intal; 1 g) was dissolved in 50 ml of water. Dilute hydrochloric acid was added to obtain a pH of 3. The precipitated white crystal was collected by filtration and washed with water, ethanol, and ether. The crystal was dried under reduced pressure to yield a white crystal of 1,3-bis(2-carboxychromon-5-yloxy)-2-hydroxypropane (600 mg, yield 66%). ¹H-NMR (DMSO-d₆) δ: 4.30 (4H, d), 4.36 (1H, t), 5.32 (1H, bs), 6.86 (2H, s), 7.11 (2H, d), 7.17 (2H, d), 7.71 (2H, t).

### (2) Synthesis of 1,3-bis(2-carboxychromon-5-yloxy)-2-hydroxypropane-immobilized resin

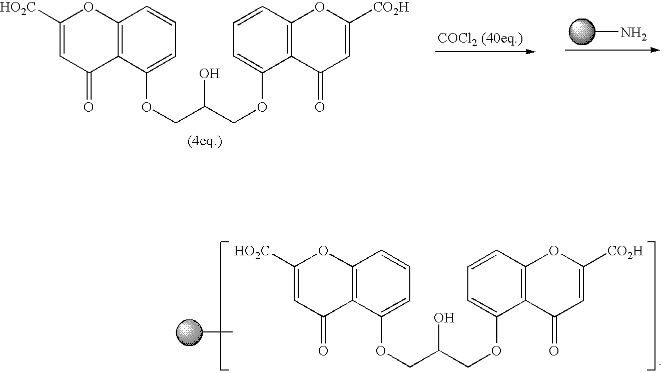

### 1) Immobilization of 1,3-bis(2-carboxychromon-5-yloxy)-2-hydroxypropane

To an acetonitrile suspension (0.5 ml) of 1,3-bis(2-carboxychromon-5-yloxy)-2-hydroxypropane (18.7 mg, 0.04 mmol), a toluene solution of phosgene (1.45 mol/1,275 μl) was added. After the reaction at room temperature for 30 minutes, the mixture was heated at 70° C. for 1 minute. The reaction solution of 1,3-bis(2-carboxychromon-5-yloxy)-2-hydroxypropane and phosgene was added to an acetonitrile suspension (0.2 ml) of TOYO-Pearl resin (TSKgel AF-amino, 100 μl, free amino group (available amino group) content 0.01 mol), and diisopropylethylamine (2.1 μl, 0.012 mmol), and the reaction was allowed to proceed at room temperature overnight. Next, the TOYO-Pearl resin was washed. The resin was washed with acetonitrile, dimethylformamide, water, and dimethylformamide five times each in this order. Residual amino groups were determined by the ninhydrin reaction, and the reaction ratio was calculated to be 43%. The resin was washed with a saturated aqueous solution of sodium hydrogen carbonate, water, and dimethylformamide five times each.

### 2) Stearic Acid and Acetyl Capping

A dimethylformamide (DMF) suspension of TOYO-Pearl resin, stearic acid (6.5 mg, 0.023 mmol), 1-(3-dimethylaminopropyl)-3-ethylcarbodiimide (4.7 μl, 0.027 mmol), and 1-hydroxybenzotriazole (HoBt; 3.6 mg, 0.027 mmol) was reacted at room temperature overnight. The TOYO-Pearl resin was washed with DMF five times. Residual amino groups were determined by the ninhydrin reaction, and the reaction ratio was calculated to be 99%. To the TOYO-Pearl resin, acetic anhydride (200 μl) and DMF (800 μl) were added. After the reaction was allowed to proceed at room temperature for 1 hour, the resin was washed with DMF five times. It was confirmed that residual amino groups were no longer macroscopically observable by the ninhydrin reaction. At this time, the reaction ratio was calculated to be 99%. Finally, the TOYO-Pearl resin was washed with a 20% aqueous solution of ethanol five times.

### Example 2

### Synthesis of Tiaramide-Immobilized Resin

### (1) Synthesis of TOYO-Pearl Resin Conjugated with Tiaramide

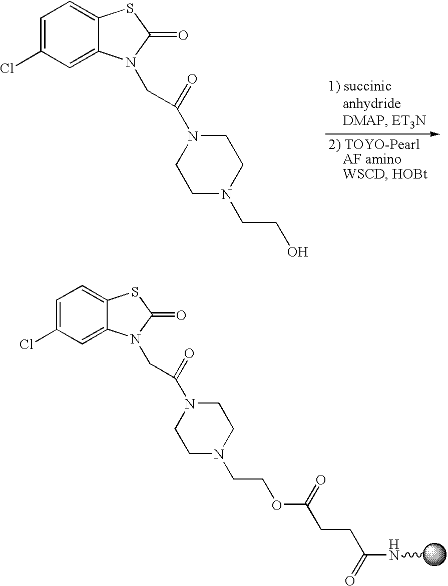

Tiaramide (36 mg, 0.1 mmol) was dissolved in 1 ml of dichloromethane, and the solution was stirred at room temperature. Succinic anhydride (12 mg, 0.12 mmol), a catalytic amount of 4(N,N-dimethylamino)pyridine (DMAP; 4 mg), and triethylamine (Et₃N, 12.1 mg, 0.12 mmol) were added, and the solution was stirred at room temperature for 3 hours. The carboxylic acid obtained was added to 5 ml of a DMF suspension of TOYO-Pearl (AF-amino) (600 μl, 60 μmol), and condensed with water-soluble carbodiimide (WSCD; 21 μl, 120 μmol) and HOBt (17.8 mg, 132 μmol). After stirring at room temperature for one day, the resin was washed with DMF five times, and the ninhydrin test was performed; as a result, the desired compound was obtained with a yield of about 97%. 5 ml of a 20% DMF solution of acetic anhydride was added, and the solution was stirred at room temperature for 30 minutes to cap the remaining amino groups with acetyl groups. The resin was washed with 20% ethanol to yield desired TOYO-Pearl resin conjugated with tiaramide.

### Example 3

### Binding Experiments

### (1) Preparation of Rat Brain Lysate

The rat brain (2.4 g) was mixed in a mixed solution A (25 mM Tris-HCl pH 8.0, 0.5% Tween 20, 300 μM DCC (24 ml; N,N-diethyldithiocarbamate sodium)) and prepared as a homogenate, which was then centrifuged at 9,000 rpm for 10 minutes. The centrifugal supernatant was collected and further centrifuged at 50,000 rpm for 30 minutes. The supernatant thus obtained was used as the lysate. Note that all experiments were performed at 4° C. or on ice.

### (2) Binding Experiments

Binding experiments were performed using the immobilizing resins with each test compound immobilized thereon, prepared in Examples 1 to 2, and the rat brain lysate prepared in Example 3(1), per the procedures shown below.

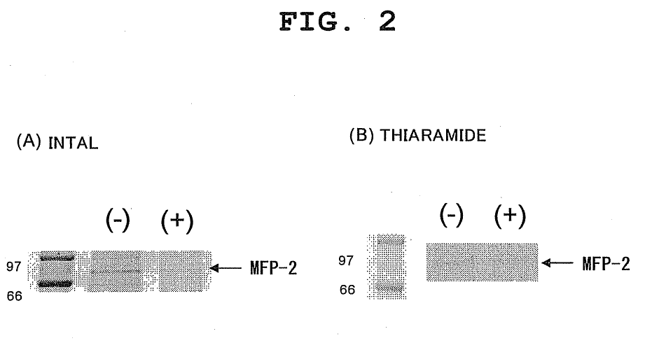

Each resin (10 μl) and lysate (1 ml) were gently shaken at 4° C. for about 1 hour. Thereafter, centrifugal operation was performed, and each supernatant was collected carefully. Then, each supernatant was again mixed with a fresh compound-bound resin (10 μl). At this time, the separated compound-bound resin was gently stored at 4° C. as the resin of the first binding experiment. After the mixture was gently stirred for about 1 hour, centrifugal operation was performed, and the supernatant was removed. Subsequently, the compound-bound resin obtained in the second binding experiment and the resin obtained in the first binding experiment were carefully washed with mixed solution A about five times each to remove substances other than the protein bound onto the resin to the maximum possible extent. To each compound-bound resin thus obtained, 25 μl of a loading buffer for SDS (nakalai Cat. NO=30566-22, sample buffer solution for electrophoresis with 2-ME (2-mercaptoethanol) (2×) for SDS PAGE) was added; this was followed by stirring at 25° C. for 10 minutes. The sample solution thus obtained was separated using a commercially available SDS gel (BioRad readyGel J, 10% SDS, cat. NO=161-J341), and the SDS gel was analyzed (FIG. 2). An electrophoregram of the sample solution comprising the protein bound onto the binding resin obtained in the first binding experiment (in FIG. 2, denoted as (−) for the sake of convenience), and an electrophoregram of the sample solution comprising the protein bound onto the binding resin obtained in the second binding experiment (in FIG. 2, denoted as (+) for the sake of convenience) were compared.

As a result, MFP-2 commonly bound to the Intal- and tiaramide-immobilized resins, and the binding was remarkably confirmed in the first binding experiment with each compound-bound resin but minimally observed in the second binding experiment; therefore, the binding was shown to be a specific binding.

Also, the subject band on the SDS gel was cut out and subjected to in-gel digestion with trypsin according to a commonly used protocol (Hisaaki Taniguchi, Miki Kikuchi, Saibo Kogaku, 21(5), p524 (2002)), after which the digested peptide fragment was examined by fingerprinting analysis using MALDI-TOF mass spectrometry to confirm that the subject protein was MFP-2. The protein search software used was Mascot.

### Example 4

### Measurement of Kd value between MFP-2 and Intal using Biacore

### Synthesis of Intal-Immobilized Chip

The SIA kit Au from Biacore Company (#BR-1004-05) was immersed in Piranha solution (25% H₂O₂, 75% H₂SO₄) in a glass dish, and washed with shaking for 3 hours. Next, this chip was washed with Milli-Q Water and ethanol, and allowed to stand overnight as immersed in an ethanol solution (1.5 mM) of 11-amino-1-undecanethiol hydrochloride (Dojindo, #A423). After this was washed with N-methylpyrrolidone (NMP), it was placed in an NMP solution containing N-Fmoc-amido-dPEG₄™-acid (50 mM), PyBop (50 mM) and diisopropylethylamine (Pr₂Net; 100 mM) and shaken for 16 hours to cause the reaction. After the mixture was washed with NMP, the reaction was allowed to proceed in an acetonitrile (MeCN) solution containing acetic acid (10 mM), HOBt (10 mM) and WSCI (water-soluble carbodiimide; 10 mM) for 5.5 hours. Furthermore, the mixture was reacted with a 20% piperidine/MeCN mixed solution for 30 minutes to achieve Fmoc elimination. Furthermore, after washing with MeCN, 70 μl of an Intal immobilization reaction solution (1.05 μM Intal, 10.5 μM HOBt, 10.5 μM WSCI) was added onto the gold membrane, and the membrane was allowed to stand overnight to immobilize the Intal.

To achieve acetyl capping after the reaction, the reaction was performed again in an MeCN solution containing acetic acid (10 mM), HOBt (10 mM), and WSCI (10 mM) for 5.5 hours to achieve capping and finish the gold membrane.

Note that control data were obtained by using a gold thin membrane chip prepared in the same manner as described above, but skipping the reaction process with the above-described Intal immobilization solution.

### <MFP-2 Expression>

An entry vector was prepared using the GATEWAY system from Invitrogen Company, and recombinant MFP-2 was expressed as a protein with N-terminal His-tag using an E. coli expression system (pDEST17). Ni-NTA purification (QIAGEN Company) and gel filtration purification (Amersham Bioscience, Superdex 200 10/300) were performed to obtain a purity of not less than 70%. The purified MFP-2 was used for measurements after buffer exchange with a running buffer (25 mM Tris-HCl, 1% CHAPS, 150 mM NaCl).

### <Measurement of Kd Value>

The Kd value was measured using Biacore 3000. The conditions were as follows: flow rate 40 μl/min; injection volume 20 μl; dissociation time 60 sec; MFP-2 concentrations 25, 12.5, and 6.25 nM.

After data were measured, differences from the control were determined using analytical software (BIACORE Company, BIA evaluation ver 4.1) and analyzed by global fitting to calculate the Kd value. As a result, the value was determined to be 0.64 nM.

From the fact above, it was demonstrated that the interaction between Intal and MFP-2 is a specific binding.

### INDUSTRIAL APPLICABILITY

Whereas ordinary NSAIDs exhibit cyclooxygenase inhibition as their essential action, a compound obtained by the screening method of the invention of this application, or the compound of the invention of this application and a pharmaceutical composition comprising the compound exhibit anti-inflammatory action and anti-allergic action by different action mechanisms. Therefore, it is possible to provide a drug that supplements the therapeutic effects of cyclooxygenase inhibitors, which produce severe adverse reactions such as gastrointestinal disorder, or that replace them.

This application is based on a patent application No. 2004-156614 filed in Japan, the contents of which are incorporated in full herein by this reference.

1. A pharmaceutical composition comprising as an active ingredient a compound that specifically binds to a protein having the amino acid sequence of SEQ ID NO:2.

2. A pharmaceutical composition comprising, as an active ingredient, a compound that specifically binds to a protein, which is characterized by having an amino acid sequence resulting from the deletion, substitution or addition of one or more amino acids in the amino acid sequence of SEQ ID NO:2 and binding to a compound of the formula (1) and/or a compound of the formula (2).

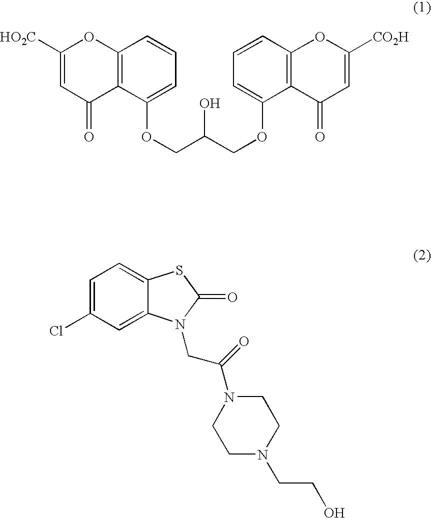

3. The pharmaceutical composition of claim 1, which is for the treatment of a disease selected from the group consisting of an inflammatory disease and an allergic disease.

4. A pharmaceutical composition comprising as an active ingredient a compound that binds specifically to MFP-2.

5. A pharmaceutical composition comprising as an active ingredient a compound that regulates the expression of MFP-2.

6. A pharmaceutical composition comprising as an active ingredient a compound that regulates the activity of MFP-2.

7. The pharmaceutical composition of claim 4, which is for the treatment of a disease selected from the group consisting of an inflammatory disease and an allergic disease.

8. A method for screening for a compound useful for the treatment of a disease selected from the group consisting of an inflammatory disease and an allergic disease, which comprises the following steps of
(1) bringing MFP-2 or a functional fragment thereof into contact with a test compound,
(2) determining whether or not the test compound specifically binds to MFP-2 or a functional fragment thereof, and
(3) selecting a test compound that specifically binds to MFP-2 or a functional fragment thereof in the step (2) above.

9. A method for screening for a compound useful for the treatment of a disease selected from the group consisting of an inflammatory disease and an allergic disease, which comprises the following steps of
(1) bringing a protein having the amino acid sequence of SEQ ID NO:2 or a functional fragment thereof into contact with a test compound,
(2) determining whether or not the test compound specifically binds to the protein or a functional fragment thereof, and
(3) selecting a test compound that specifically binds to the protein or a functional fragment thereof in the step (2) above.

10. A method for screening for a compound useful for the treatment of a disease selected from the group consisting of an inflammatory disease and an allergic disease, which comprises the following steps of
(1) bringing a protein, which is characterized by having an amino acid sequence resulting from the deletion, substitution or addition of one or more amino acids in the amino acid sequence of SEQ ID NO:2 and binding to a compound of the formula (1) and/or a compound of the formula (2), or a functional fragment thereof, into contact with a test compound, 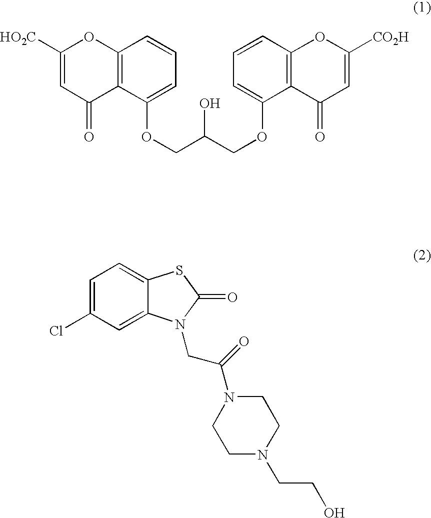
(2) determining whether or not the test compound specifically binds to the protein or a functional fragment thereof, and
(3) selecting a test compound that specifically binds to the protein or a functional fragment thereof in the step (2) above.

11. A compound useful for the treatment of a disease selected from the group consisting of an inflammatory disease and an allergic disease, obtained by the screening method of claim 8.

12. A compound represented by the general formula (1) or the general formula (II) or a pharmaceutically acceptable salt thereof: 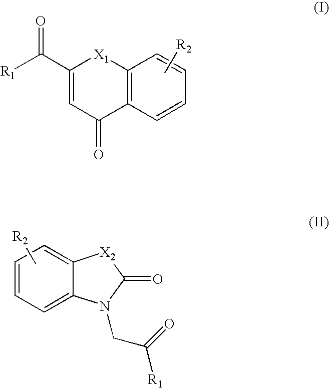
wherein X₁ is O, NH, NR′″, S, SO or SO₂;
X₂ is O, NH, NR′″, S, CH₂CH₂— or —CH═CH—;
R₁ is H, OH, OR′, NH₂, NHR′ or NR′R″;
R₂ is an optionally substituted saturated or unsaturated chain-structured hydrocarbon group, an optionally substituted alkoxy group, an optionally substituted amino group, an optionally substituted carboxyl group, an optionally substituted amide group or a halogen atom;
R′, R″ and R′″ are the same or different and each is an optionally substituted saturated or unsaturated chain-structured hydrocarbon group;
with the proviso that when R₁ is NR′R″, R′ and R″ may bind together to form a ring in cooperation with the adjoining nitrogen tom), atom, and
with the proviso that the compound is not a compound represented by formula (1) or formula (2).

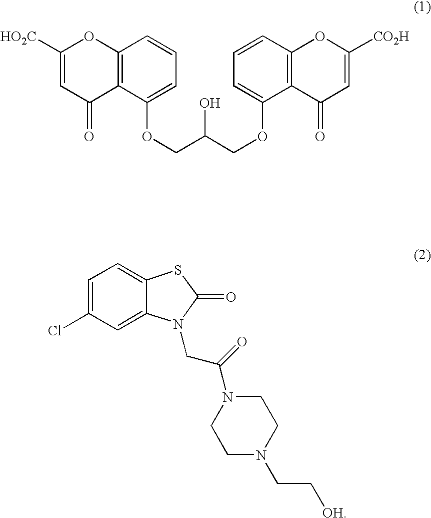

13. A pharmaceutical composition comprising as an active ingredient the compound of claim 12 or a pharmaceutically acceptable salt thereof.

14. The pharmaceutical composition of claim 13, which is for the treatment of a disease selected from the group consisting of an inflammatory disease and an allergic disease.

15. The pharmaceutical composition of claim 2, which is for the treatment of a disease selected from the group consisting of an inflammatory disease and an allergic disease.

16. The pharmaceutical composition of claim 5, which is for the treatment of a disease selected from the group consisting of an inflammatory disease and an allergic disease.

17. The pharmaceutical composition of claim 6, which is for the treatment of a disease selected from the group consisting of an inflammatory disease and an allergic disease

18. A compound useful for the treatment of a disease selected from the group consisting of an inflammatory disease and an allergic disease, obtained by the screening method of claim 9.

19. A compound useful for the treatment of a disease selected from the group consisting of an inflammatory disease and an allergic disease, obtained by the screening method of claim 10.


SEQUENCE LISTING (6 sequences)

SEQ ID NO: 1 - DNA, 2322 bp, Rattus norvegicus
  CDS (28)..(2232)
gtgtgtgcgtggtgcaggatagactcaatgtcgcctctgaggttcgacgggcgtgtgggcctggtcaccggcgccgggggagggttgggcagagcttatgggctggcttttgcagaaagaggagcattagttgttgtgaatgacttaggaggggacttcaaaggcgttgggaaaggctcttctgccgcagacaaggtcgtggaagaaataagaaggagaggcgggaaagcggtggccaattacgattcagtcgaagcaggcgagaagcttgtgaagacagcactggacacattcggcagaatagatgttgtggtgaacaatgctgggatcctgagggacccttccttctctaggataagtgatgaagactgggatataattcaaagagttcatttgcggggctccttccaagtgacccgggcagcatgggatcatatgaagaagcagaattatggaagaatcattatgacggcctcagcttctggaatatacggcaactttggccaggcaaattatagtgctgcaaagctgggccttctgggtctcgccaatactctcgtgattgaaggcaggaagaacaacattcattgtaacaccattgccccaaacgctgggtcacggatgacagagacggtgatgccagaagacctcgttgaagccctgaagccagagtatgtggcaccgctggtcctttggctttgccatgagagctgtgaggaaaatggtggcttgtttgaggttggagcaggatggattggaaaattgcgctgggagaggaccctgggagccattgtcaggaagcggaatcagcccatgactcccgaggcagtgagggacaactgggtgaagatctgtgacttcagcaatgccagcgagccgaagagcattcaagagtccacaggtggtataatcgaagttttacataaaatagattcagaaggaatctcacaaaatcacaccggtcaagtggcatctgcagatgcatcaggatttgctggcgtcgttggccacaaacttccttcattttcttcttcatatacggaactgcagtgcattatgtatgccctcggagtaggagcttcagtcaaaaatccaaaggacttgaagtttgtttatgaagggagtcctgacttctcctgtttgcctacaattggagtcattgtcgctcagaagtccttgatgagtggaggcttagcagaggttcctgggctgtcaatcaactttgcaaaggttcttcatggggagcagtacttggagttgtataagccacttccccgatcaggggaattaaaatgtgaagcagttattgctgacatcctggataaaggctctggcatagtgattgttatggacgtctattcttattctggcaaggaacttatatgctataatcagttctctgtcttcgttgttggctctggaggctttggtggaaaacggacatcagaaaaactcaaagcagctgtagccgtaccaagtcggcctccagatgctgtactgagagataccacttcagtgaatcaggccgctctgtaccgcctcagtggagactcgaatcctttacacattgacccgagctttgcaggcattgccggttttgagaaacccatattacacggattatgtacttttgggttttctgcaaggcatgttttacagcagtttgcggataatgatgtgtcaagattcaaggccattaaggttcgttttgccaaaccagtgtatccaggacaaactctacaaactgagatgtggaaggaaggaaacagaattcattttcaaaccaaggtccaagagactggagacattgtcatttccaatgcatatgtggatcttgttcctacatctggagtttccgctcagacaccttctgagggtggagcactgcagagtgctcttgtatttggggaaataggtcgacgcctcaaggatgttggacgtgaggtggtaaagaaagtaaatgctgtatttgaatggcatatcacgaaaaatgggaatgttgcagccaagtggaccattgacctgaagaacggctctggagaggtttaccaaggccctgccaaaggctctgctgacacgaccatcacaatttctgatgaggatttcatggaagtggtcctgggcaagcttaacccacagaatgccttcttcagtggcagactgaaggcccgaggaaacatcatgctgagccagaagctacagatgattctgaaagactatgccaagctctgaaggacccactgcgtgctttaataaaaccagaatcattacgttctgtctacgcagtcatgctccagccttctttgaaacgatccacggtaa

SEQ ID NO: 2 - protein, 734 aa, Rattus norvegicus
MSPLRFDGRVGLVTGAGGGLGRAYGLAFAERGALVVVNDLGGDFKGVGKGSSAADKVVEEIRRRGGKAVANYDSVEAGEKLVKTALDTFGRIDVVVNNAGILRDPSFSRISDEDWDIIQRVHLRGSFQVTRAAWDHMKKQNYGRIIMTASASGIYGNFGQANYSAAKLGLLGLANTLVIEGRKNNIHCNTIAPNAGSRMTETVMPEDLVEALKPEYVAPLVLWLCHESCEENGGLFEVGAGWIGKLRWERTLGAIVRKRNQPMTPEAVRDNWVKICDFSNASEPKSIQESTGGIIEVLHKIDSEGISQNHTGQVASADASGFAGVVGHKLPSFSSSYTELQCIMYALGVGASVKNPKDLKFVYEGSPDFSCLPTIGVIVAQKSLMSGGLAEVPGLSINFAKVLHGEQYLELYKPLPRSGELKCEAVIADILDKGSGIVIVMDVYSYSGKELICYNQFSVFVVGSGGFGGKRTSEKLKAAVAVPSRPPDAVLRDTTSVNQAALYRLSGDSNPLHIDPSFAGIAGFEKPILHGLCTFGFSARHVLQQFADNDVSRFKAIKVRFAKPVYPGQTLQTEMWKEGNRIHFQTKVQETGDIVISNAYVDLVPTSGVSAQTPSEGGALQSALVFGEIGRRLKDVGREVVKKVNAVFEWHITKNGNVAAKWTIDLKNGSGEVYQGPAKGSADTTITISDEDFMEVVLGKLNPQNAFFSGRLKARGNIMLSQKLQMILKDYAKL

SEQ ID NO: 3 - DNA, 2593 bp, Homo sapiens
  CDS (49)..(2259)
ggccagcgcgtctgcttgttcgtgtgtgtgtcgttgcaggccttattcatgggctcaccgctgaggttcgacgggcgggtggtactggtcaccggcgcgggggcaggattgggccgagcctatgccctggcttttgcagaaagaggagcgttagttgttgtgaatgatttgggaggggacttcaaaggagttggtaaaggctccttagctgctgataaggttgttgaagaaataagaaggagaggtggaaaagcagtggccaactatgattcagtggaagaaggagagaaggttgtgaagacagccctggatgcttttggaagaatagatgttgtggtcaacaatgctggaattctgagggatcgttcctttgctaggataagtgatgaagactgggatataatccacagagttcatttgcggggttcattccaagtgacacgggcagcatgggaacacatgaagaaacagaagtatggaaggattattatgacttcatcagcttcaggaatatatggcaactttggccaggccaattatagtgctgcaaagttgggtcttctgggccttgcaaattctcttgcaattgaaggcaggaaaagcaacattcattgtaacaccattgctcctaatgcgggatcacggatgactcagacagttatgcctgaagatcttgtggaagccctgaagccagagtatgtggcacctcttgtcctttggctttgtcacgagagttgtgaggagaatggtggcttgtttgaggttggagcaggatggattggaaaattacgctgggagcggactcttggagctattgtaagacaaaagaatcacccaatgactcctgaggcagtcaaggctaactggaagaagatctgtgactttgagaatgccagcaagcctcagagtatccaagaatcaactggcagtataattgaagttctgagtaaaatagattcagaaggaggagtttcagcaaatcatactagtcgtgcaacgtctacagcaacatcaggatttgctggagctattggccagaaactccctccattttcttatgcttatacggaactggaagctattatgtatgcccttggagtgggagcgtcaatcaaggatccaaaagatttgaaatttatttatgaaggaagttctgatttctcctgtttgcccaccttcggagttatcataggtcagaaatctatgatgggtggaggattagcagaaattcctggactttcaatcaactttgcaaaggttcttcatggagagcagtacttagagttatataaaccacttcccagagcaggaaaattaaaatgtgaagcagttgttgctgatgtcctagataaaggatccggtgtagtgattattatggatgtctattcttattctgagaaggaacttatatgccacaatcagttctctctctttcttgttggctctggaggctttggtggaaaacggacatcagacaaagtcaaggtagctgtagccatacctaatagacctcctgatgctgtacttacagataccacctctcttaatcaggctgctttgtaccgcctcagtggagactggaatcccttacacattgatcctaactttgctagtctagcaggttttgacaagcccatattacatggattatgtacatttggattttctgccaggcgtgtgttacagcagtttgcagataatgatgtgtcaagattcaaggcaattaaggctcgttttgcaaaaccagtatatccaggacaaactctacaaactgagatgtggaaggaaggaaacagaattcattttcaaaccaaggtccaagaaactggagacattgtcatttcaaatgcatatgtggatcttgcaccaacatctggtacttcagctaagacaccctctgagggcgggaagcttcagagtacctttgtatttgaggaaataggacgccgcctaaaggatattgggcctgaggtggtgaagaaagtaaatgctgtatttgagtggcatataaccaaaggcggaaatattggggctaagtggactattgacctgaaaagtggttctggaaaagtgtaccaaggccctgcaaaaggtgctgctgatacaacaatcatactttcagatgaagatttcatggaggtggtcctgggcaagcttgaccctcagaaggcattctttagtggcaggctgaaggccagagggaacatcatgctgagccagaaacttcagatgattcttaaagactacgccaagctctgaagggcacactacactattaataaaaatggaatcattaaatactctcttcacccaaatatgcttgattattctgcaaaagtgattagaactaagatgcaggggaaattgcttaacattttcagatatcagataactgcagattttcattttctactaattttcatgtatcattatttttacaaggaactatatataagctagcacatgattatccttctgttcttagatctgtatcttcataataaaaaattttgcccaagtcctgtttccttagaatttgtgatagcattgataagttgaaaggaaaattaaatcaataaaggcctttgatacc

SEQ ID NO: 4 - protein, 736 aa, Homo sapiens
MGSPLRFDGRVVLVTGAGAGLGRAYALAFAERGALVVVNDLGGDFKGVGKGSLAADKVVEEIRRRGGKAVANYDSVEEGEKVVKTALDAFGRIDVVVNNAGILRDRSFARISDEDWDIIHRVHLRGSFQVTRAAWEHMKKQKYGRIIMTSSASGIYGNFGQANYSAAKLGLLGLANSLAIEGRKSNIHCNTIAPNAGSRMTQTVMPEDLVEALKPEYVAPLVLWLCHESCEENGGLFEVGAGWIGKLRWERTLGAIVRQKNHPMTPEAVKANWKKICDFENASKPQSIQESTGSIIEVLSKIDSEGGVSANHTSRATSTATSGFAGAIGQKLPPFSYAYTELEAIMYALGVGASIKDPKDLKFIYEGSSDFSCLPTFGVIIGQKSMMGGGLAEIPGLSINFAKVLHGEQYLELYKPLPRAGKLKCEAVVADVLDKGSGVVIIMDVYSYSEKELICHNQFSLFLVGSGGFGGKRTSDKVKVAVAIPNRPPDAVLTDTTSLNQAALYRLSGDWNPLHIDPNFASLAGFDKPILHGLCTFGFSARRVLQQFADNDVSRFKAIKARFAKPVYPGQTLQTEMWKEGNRIHFQTKVQETGDIVISNAYVDLAPTSGTSAKTPSEGGKLQSTFVFEEIGRRLKDIGPEVVKKVNAVFEWHITKGGNIGAKWTIDLKSGSGKVYQGPAKGAADTTIILSDEDFMEVVLGKLDPQKAFFSGRLKARGNIMLSQKLQMILKDYAKL

SEQ ID NO: 5 - DNA, 2468 bp, Mus musculus
  CDS (13)..(2220)
caggctgagctcatggcttcccccctgaggttcgacgggcgtgtggtcttggtcaccggccccgggggaggattgggccgagcttacgccctggcgtttgcagaaagaggagcattagtcattgtgaacgacttaggaggggacttcaagggaattggtaaaggctcctctgctgcagacaaggttgtggcagagataagaaggaaaggcggaaaagcagtggccaattacgattcagttgaagcaggcgagaagcttgtgaagacggcactggacacatttggcagaatagacgttgtggtcaacaatgctggaatcctgagggaccgttccttctccaggataagtgatgaagactgggatataattcatagagttcatttgcggggctccttccaagtgacccgggcagcatgggaccatatgaagaaacagaattatggaagaatccttatgacttcctcagcttctggaatatatggcaactttggccaggcgaattatagtgctgcaaagctgggcattctgggtctctgcaatactctcgccattgaaggcaggaagaacaacattcattgcaacaccattgcccccaacgctgggtcacggatgacggagactgtgttgccggaagatcttgttgaagccctgaagccagagtatgtggcccctctggtgctttggctttgccatgagagctgtgaggaaaatggtggcctatttgaggttggagcaggatggattggaaaattgcgctgggagaggaccctgggcgccatcgtcagaaagcggaatcagcccatgactcccgaggcagtgagggacaactgggagaagatctgtgacttcagcaatgccagcaagccgcagaccattcaagaatcaacaggtggtatagtcgaagttttacataaggtagattcagaaggaatctcaccaaaccgtaccagtcacgcggcacctgcagccacgtcaggattcgttggtgctgttggccataaacttccttcattttcttcttcgtatacggagctgcagagtattatgtatgccctcggagtgggagcgtcagtcaaaaatccaaaggatttgaagtttgtttatgaaggcagtgctgacttctcctgtttgcccaccttcggagtcattgtcgctcagaagtccatgatgaatggagggctggcagaggttcctgggctgtcattcaactttgcaaaggctcttcacggggagcagtacttggagctgtataagccacttcttcgatcaggagaattaaaatgtgaagcagttattgctgacatcctggataaaggctctggcgtagtgattgttatggacgtctattcttattctgggaaggaacttatatgctataatcagttctctgtctttgttgttggctctgggggctttggtggaaaacggacatcagaaaaactcaaagcagctgtagctgtaccaaatcgacctccagatgctgtactgagagatgccacctcactgaatcaggccgcgctgtaccgcctcagcggagactggaatcctctacacattgacccggactttgcgagcgttgccggttttgagaagcccatattacatggactatgtacctttggattttctgcaaggcatgttttacagcagtttgcagataatgatgtatcaagattcaaggcgattaaggttcgttttgccaaaccagtgtatccaggacagactctacaaactgagatgtggaaggaaggaaacagaattcattttcaaaccaaggtccacgagactggagatgttgtcatttcaaatgcgtacgtggatctcgtgcctgcatctggagtttcaacccagacaccttcagagggtggagagctccagagtgctcttgtgtttggggagataggccgccgcctcaagagtgttggccgtgaggtggtaaagaaagcgaatgctgtgtttgaatggcatatcacgaaaggtgggactgttgcagccaagtggaccattgacctgaagagcggctcaggggaggtgtaccaaggccccgcaaagggctctgctgatgtgaccatcatcatttccgatgaggattttatggaagtggtcttcggcaagcttgacccacagaaggccttcttcagtggcaggctgaaggccagagggaacatcatgctgagccagaaactacagatgattcttaaagactatgccaagctctgaagggaacccactgtgtgctgttaaaggagtcaataattaaatactgtctacccagctgagccgcagccttctgcgatccacaggagtgtgcaggagaaatcgcttcacatttccagattcagataacttgcatattttcattttctactaatttttcacatatttttacaaggaactgtaatctaggtagcaaaataatcattctgttcatagatctgtatcttaataaaaaaaatcaaccaaaaacc

SEQ ID NO: 6 - protein, 735 aa, Mus musculus
MASPLRFDGRVVLVTGPGGGLGRAYALAFAERGALVIVNDLGGDFKGIGKGSSAADKVVAEIRRKGGKAVANYDSVEAGEKLVKTALDTFGRIDVVVNNAGILRDRSFSRISDEDWDIIHRVHLRGSFQVTRAAWDHMKKQNYGRILMTSSASGIYGNFGQANYSAAKLGILGLCNTLAIEGRKNNIHCNTIAPNAGSRMTETVLPEDLVEALKPEYVAPLVLWLCHESCEENGGLFEVGAGWIGKLRWERTLGAIVRKRNQPMTPEAVRDNWEKICDFSNASKPQTIQESTGGIVEVLHKVDSEGISPNRTSHAAPAATSGFVGAVGHKLPSFSSSYTELQSIMYALGVGASVKNPKDLKFVYEGSADFSCLPTFGVIVAQKSMMNGGLAEVPGLSFNFAKALHGEQYLELYKPLLRSGELKCEAVIADILDKGSGVVIVMDVYSYSGKELICYNQFSVFVVGSGGFGGKRTSEKLKAAVAVPNRPPDAVLRDATSLNQAALYRLSGDWNPLHIDPDFASVAGFEKPILHGLCTFGFSARHVLQQFADNDVSRFKAIKVRFAKPVYPGQTLQTEMWKEGNRIHFQTKVHETGDVVISNAYVDLVPASGVSTQTPSEGGELQSALVFGEIGRRLKSVGREVVKKANAVFEWHITKGGTVAAKWTIDLKSGSGEVYQGPAKGSADVTIIISDEDFMEVVFGKLDPQKAFFSGRLKARGNIMLSQKLQMILKDYAKL